(19) United States
(12) Patent Application Publication
(10) Pub. No.: US 2022/0148734 A1
(43) Pub. Date: May 12, 2022

(54) BLOOD CELL-FREE DNA-BASED METHOD FOR PREDICTING PROGNOSIS OF LIVER CANCER TREATMENT

(71) Applicants: GREEN CROSS GENOME CORPORATION, Gyeonggi-do (KR); NATIONAL CANCER CENTER, Gyeonggi-do (KR); THE ASAN FOUNDATION, Seoul (KR); UNIVERSITY OF ULSAN FOUNDATION FOR INDUSTRY COOPERATION, Ulsan (KR)

(72) Inventors: Baek-Yeol RYOO, Seoul (KR); Sook Ryun PARK, Seoul (KR); Eun Hae CHO, Gyeonggi-do (KR); Junnam LEE, Gyeonggi-do (KR); Sun-Young KONG, Gyeonggi-do (KR); Min Kyeong KIM, Gyeonggi-do (KR)

(21) Appl. No.: 17/429,343

(22) PCT Filed: Feb. 19, 2020
(86) PCT No.: PCT/KR2020/002359

§ 371 Date: Aug. 8, 2021

(30) Foreign Application Priority Data
Feb. 19, 2019 (KR) 10-2019-0019315

(51) Int. Cl. G16H 50/30; G16B 30/10; G16B 40/00; G16H 50/20; G16H 50/50

(52) U.S. Cl. CPC G16H 50/30; G16B 30/10; G16H 50/50; G16H 50/20; G16B 40/00

(57) ABSTRACT

The present invention relates to a blood cell-free DNA-based method for predicting the prognosis of liver cancer treatment. A method for predicting the prognosis of liver cancer, according to the present invention, uses next generation sequencing (NGS) so as to increase the accuracy of prognosis prediction of a liver cancer patient and also increase the accuracy of prognosis prediction based on a very low concentration cell-free DNA of which detection has been difficult, thereby increasing the commercial utilization thereof. Therefore, the method of the present invention is useful for determining the prognosis of a liver cancer patient.

### TECHNICAL FIELD

The present invention relates to a method for determining the prognosis of liver cancer treatment based on blood cell-free DNA, and more specifically to a method for predicting the prognosis of liver cancer treatment by extracting cell-free DNA (cfDNA) from a biological sample to obtain sequence information and then performing normalization and regression analysis in the chromosomal region.

### BACKGROUND ART

Primary liver cancer is the third most common cause of cancer death worldwide, and the incidence thereof is continually increasing (Ferlay J. et al., Int. J. Cancer Vol. 136:E359-86, 2015). Liver cancer cases accounted for 15,757 cancer cases, corresponding to 7.3% of the total of 214,701 cancer cases that occurred in Korea in 2015, ranking the sixth most common of all forms of cancer, and had the second highest cancer mortality rate. The incidence of liver cancer depending on age was the highest for those in their 50s, with 27.1%, and was 26.0% and 23.9% for those in their 60s and 70s, respectively. Among primary liver cancers, hepatocellular carcinoma is the main histological subtype which accounts for 85 to 90% of all liver cancer. The main cause of the development of hepatocellular carcinoma is infection with hepatitis B and C virus. In addition to the hepatitis virus, long-term alcohol consumption and cirrhosis are also known as risk factors for liver cancer. The results of research have reported that hepatocellular carcinoma was found within 5 years in 8% of patients with alcoholic cirrhosis and 4% of patients with cirrhosis, and it is known that as cirrhosis is severe and with increasing age, the risk of developing liver cancer increases (Fattovich G. et al., Gastroenterology), Vol. 127:S35-50, 2004).

Cancer is caused by failure of normal regulation of cell division due to gene mutations accumulated in cells. For this reason, cancer cells are characterized by frequent chromosomal abnormalities such as deletion, duplication, and translocation. In particular, it is known that activation of oncogenes or inactivation of tumor suppressor genes due to chromosomal abnormalities have a great influence on the incidence of cancer. The onset of liver cancer is highly correlated with the overlap of chromosomes 1, 7, 8, 17, 20 and deletion of chromosomes 4, 8, 13, 16, and 17 (Zhou C. et al., Sci Rep. 2017 Vol. 7(1):10570). In particular, somatic copy number alteration (SCNA) in liver cancer patients is frequently found in p53 signaling (TP53, CDKN2A), Wnt/β-catenin pathway (CTNNB1, AXIN1) and chromosomal remodeling (ARID1A, ARID1B, ARID2)-related genes and telomerase maintenance-related TERT genes (Ng CKY, et al., Front. Med. (Lausanne). 2018 Vol. 5:78). These genes are genes related to the regulation of cell cycle and cell growth, and studies showing the association between these genes and the development of liver cancer have been reported (Ju-Seog Lee, Clin Mol Hepatol. 2015 Vol. 21(3): 220-229). As studies on the mechanism of occurrence of cancer due to chromosomal abnormalities are conducted, efforts to use the same as an index for diagnosis and prognosis of cancer are continuing (Parker B. C. and Zhang W., Chin. J. Cancer. Vol. 11:594-603. 2013).

Furthermore, recently, studies have been conducted to detect chromosomal abnormalities using cell-free DNA (cfDNA), which is present in plasma through necrosis, apoptosis and secretion of cells, based on liquid biopsy technology. In particular, blood-cell-free DNA derived from tumor cells includes tumor-specific chromosomal abnormalities and mutations that are not found in normal cells, and has the advantage of reflecting the current state of tumors due to the short half-life thereof of 2 hours. In addition, blood-cell-free DNA is in the spotlight as a tumor-specific biomarker in various cancer-related fields such as diagnosis, monitoring and prognosis of cancer because collection thereof is noninvasive and can be performed repeatedly. With recent advances in molecular diagnostic technology, research has reported that it is possible to detect tumor-specific chromosomal abnormalities in blood-cell-free DNA of cancer patients through digital karyotyping and PARE analysis, and the results of research have clinically confirmed the same (Leary R. J. et al., Sci. Transl. Med. Vol. 4, Issue 162. 2012).

According to research by Faye R. Harris in 10 ovarian cancer patients, microdeletions identified in the patient's cancer tissue DNA were analyzed from ctDNA obtained before and after surgery (Harris F R et al., Sci Rep. Vol. 6: 29831. 2016). As a result, microdeletion was detected in 8 patients before surgery and in 3 patients exhibiting recurrence, out of 8 patients after surgery. This indicates that the detection of microdeletion of cell-free DNA in blood was clinically significant and that tumor-specific chromosomal abnormalities were reflected in cell-free DNA in the blood.

In addition, Daniel G. Stover analyzed tissue-specific CNA through cfDNA in 164 metastatic TNBC (triple-negative breast cancer) patients (Stover D G. et al., J. Clin. Oncol. Vol. 36(6):543-553). The result showed that the increase in the number of copies of specific genes such as NOTCH2, AKT2 and AKT3 was higher in metastatic TNBC than in primary TNBC, and the survival rate of metastatic TNBC patients with overlapping 18q11 and 19p13 chromosomes was statistically significantly lower.

Accordingly, against this technical background, as a result of extensive efforts to develop a method for determining the prognosis of liver cancer based on cell-free DNA in the blood, the present inventors found that when performing normalization correction and regression analysis on blood-cell-free DNA chromosomal region and concentration, the prognosis of liver cancer patients can be determined with high sensitivity. Based on this finding, the present invention was completed.

### [Abstract]

Therefore, the present invention has been made in view of the above problems, and it is one object of the present invention to provide a method of determining the prognosis of liver cancer based on cell-free DNA (cfDNA).

It is another object of the present invention to provide a device for determining the prognosis of liver cancer.

It is another object of the present invention to provide a computer-readable medium including instructions designed to be executed by a processor for determining the prognosis of liver cancer using the method.

It is another object of the present invention to provide a method of providing information for determining the prognosis of liver cancer including the method.

In accordance with one aspect of the present invention, the above and other objects can be accomplished by the provision of a method of determining a prognosis of liver cancer based on cell-free DNA (cfDNA), the method including: a) obtaining reads (sequence information) of the cell-free DNA isolated from a biological sample; b) aligning the reads to a reference genome database of a reference group; c) detecting a quality of the aligned reads and selecting only reads having a quality equal to or higher than a cut-off value; d) segmenting the reference genome into predetermined bins, and detecting and normalizing amounts of the selected reads in the respective bins; e) calculating a mean and a standard deviation of normalized reads matched to each bin of the reference group and then calculating a Z score from normalized values in step d); f) segmenting chromosome using the Z score and calculating an I score; and g) determining that a prognosis of liver cancer is bad when the resulting I score is higher than a cut-off value.

In accordance with another aspect of the present invention, provided is a device for determining a prognosis of liver cancer based on cell-free DNA (cfDNA), the device including: a decoder for decoding reads (sequence information) of cell-free DNA isolated from a biological sample; an aligner for aligning the decoded reads to a reference genome database of a reference group; a quality controller for selecting only reads having a quality equal to or higher than a cut-off value from the aligned reads; and a determiner for calculating a Z score through comparison of selected reads with a reference group sample, calculating an I score based on the Z score and determining that the prognosis of liver cancer is bad when the I score is higher than a cut-off value.

In accordance with another aspect of the present invention, provided is a computer-readable medium including an instruction configured to be executed by a processor for determining a prognosis of liver cancer, the computer-readable medium including: a) obtaining reads (sequence information) of cell-free DNA isolated from a biological sample; b) aligning the reads to a reference genome database of a reference group; c) detecting a quality of the aligned reads and selecting only reads having a quality equal to or higher than a cut-off value; d) segmenting the reference genome into predetermined bins, and detecting and normalizing amounts of the selected reads in the respective bins; e) calculating a mean and a standard deviation of normalized reads matched to each bin of the reference group and then calculating a Z score from normalized values in step d); f) segmenting chromosome using the Z score and calculating an I score; and g) determining that a prognosis of liver cancer is bad when the resulting I score is higher than a cut-off value.

In accordance with another aspect of the present invention, provided is a method of providing information for determining the prognosis of liver cancer including the method.

### DESCRIPTION OF DRAWINGS

FIG. 1 is an overall flow chart showing the determination of prognosis of liver cancer based on cfDNA according to the present invention.

FIG. 2 is a schematic diagram showing the result of calibration of the number of sequencing reads before and after GC calibration using a LOESS algorithm during the process of quality control (QC) of read data.

FIG. 3 shows the result of confirming the difference in blood cell-free DNA concentration between a normal subject and a liver cancer patient.

FIG. 4 shows the result of evaluation of the progression of liver cancer and survival according to the cell-free DNA concentration in the blood.

FIG. 5 shows the result of a determination of prognosis for progression of liver cancer and survival according to the method of the present invention.

FIG. 6 shows the result of the determination of prognosis on the survival of liver cancer patients in each of groups classified on the basis of an I score according to the present invention.

FIG. 7 shows the result of a determination of prognosis on the progression of liver cancer in each of groups classified on the basis of an I score according to the present invention.

FIG. 8 shows the result confirming the correlation between the concentration of cell-free DNA in the blood and the I score of the present invention.

### BEST MODE

Unless defined otherwise, all technical and scientific terms used herein have the same meanings as appreciated by those skilled in the field to which the present invention pertains. In general, the nomenclature used herein is well-known in the art and is ordinarily used.

It was found in the present invention that sequence analysis data (reads) obtained from a liver cancer patient sample was normalized and organized based on a cut-off value, chromosome was segmented into predetermined bins, the amount of reads in each bin was normalized, a Z score was calculated through comparison with a reference group sample, chromosome was segmented again based on the calculated Z score, an I score was calculated based thereon, and the prognosis was determined to be bad when the I-score is higher than 1637, and was determined to be good when the I-score is not higher than 1637. Specifically, the risk groups for death from liver cancer or progression thereof could be classified and identified depending on the range of the I score. More specifically, the case where the I score is 1638 to 3012 is classified as a moderate risk group, the case where the I score is 3013 to 7448 and the case where the I score is 7449 to 13672, are classified as a high risk group, and the case where the I score is 13673 to 28520 is classified as an ultra-high risk group.

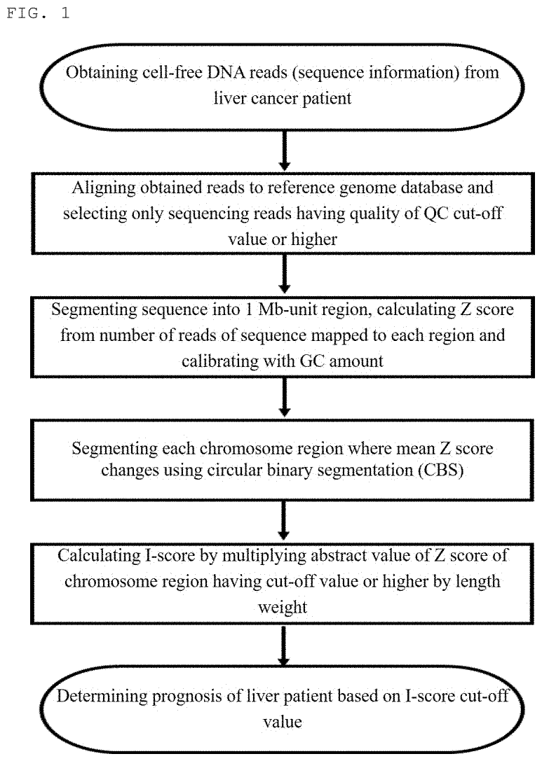

That is, in an embodiment of the present invention, developed was a method of determining the prognosis of liver cancer including sequencing DNA extracted from the blood of 14 normal subjects and 151 liver cancer patients, controlling quality using the LOESS algorithm, segmenting chromosome into predetermined bins to normalize the amount of reads matched to each bin with a GC ratio, calculating the mean and standard deviation of the reads matched to each bin in a normal sample, calculating a Z score with the normalized value, segmenting an area of chromosome where the Z score rapidly changes again based thereon, calculating an I-score using the same, and determining that the prognosis of the liver cancer patient is bad when the I-score is higher than 1637 (FIG. 1).

As used herein, the term “read” refers to one nucleic acid fragment obtained by analyzing sequence information using any of a variety of methods known in the art. Therefore, the term “read” has the same meaning as the term “sequence information” in that they both refer to sequence information results obtained through a sequencing process.

As used herein, the term “determination of prognosis” has the same meaning as the term “prognosis”, and refers to an act of predicting the course and outcome of a disease in advance. More specifically, the term “determination of prognosis” is interpreted to mean any action that predicts the course of a disease after treatment in comprehensive consideration of the physiological or environmental state of a patient, and the course of the disease after treatment of the disease may vary depending on the physiological or environmental state of the patient.

For the purposes of the present invention, the determination of prognosis can be interpreted as an act of predicting the progression of a disease after treatment of liver cancer and predicting the risk of progression of cancer, recurrence of cancer, and/or metastasis of cancer. For example, the expression “good prognosis” or “prognosis is good” means that the risk index of progression of cancer, recurrence of cancer and/or metastasis of cancer in a liver cancer patient after liver cancer treatment is lower than 1 and that the liver cancer patient is more likely to survive, and is also expressed as “positive prognosis”. The expression “bad prognosis” means that the risk of progression of cancer, recurrence of cancer and/or metastasis of cancer in a liver cancer patient after liver cancer treatment is higher than 1, and that the liver cancer patient is more likely to die, and is also expressed as “negative prognosis”.

As used herein, the term “risk index” refers to an odds ratio, a hazard ratio, or the like regarding the probability that progression, recurrence, and/or metastasis of cancer will occur in a patient after treatment of liver cancer.

In one aspect, the present invention is directed to a method of determining a prognosis of liver cancer based on cell-free DNA (cfDNA), the method including:

a) obtaining reads (sequence information) of cell-free DNA isolated from a biological sample;

b) aligning the reads to a reference genome database of a reference group;

c) detecting a quality of the aligned reads and selecting only reads having a quality equal to or higher than a cut-off value;

d) segmenting the reference genome into predetermined bins, and detecting and normalizing amounts of the selected reads in the respective bins;

e) calculating a mean and a standard deviation of normalized reads matched to each bin of the reference group and then calculating a Z score from normalized values in step d);

f) segmenting chromosome using the Z score and calculating an I score; and

g) determining that a prognosis of liver cancer is bad when the resulting I score is higher than a cut-off value.

In the present invention,

step a) is carried out by a process including:

(a-i) removing proteins, fats and other residues from the isolated cell-free DNA using a salting-out method, a column chromatography method, or a bead method to obtain purified nucleic acids;

(a-ii) producing a single-end-sequencing or paired-end-sequencing library from the purified nucleic acids;

(a-iii) applying the produced library to a next-generation sequencer; and

(a-iv) obtaining reads of the nucleic acids from the next-generation sequencer.

The method may further include, between the steps (a-i) and (a-ii), randomly fragmenting the nucleic acids purified in the step (a-i) by an enzymatic digestion, pulverization or HydroShear method to produce the single-end sequencing or paired-end sequencing library.

In the present invention, step a) of obtaining the reads may include obtaining the isolated cell-free DNA through full-length genome sequencing with a depth of 1 million to 100 million reads.

As used herein, the term “reference group” refers to a reference group that can be used for comparison, like a standard nucleotide sequence database, and means a population of humans who do not currently have a specific disease or condition. In the present invention, the standard nucleotide sequence in the standard genome database of the reference group may be reference genome registered with a public health institution such as NCBI.

In the present invention, the next-generation sequencer may be a Hiseq system produced by Illumina Inc., a Miseq system produced by Illumina Inc., a genome analyzer (GA) produced by Illumina Inc., 454 FLX produced by Roche Applied Science, SOLiD system produced by Applied Biosystems Company, or the Ion Torrent system produced by Life Technologies Company, but is not limited thereto.

In the present invention, the alignment may be performed using the BWA algorithm and the Hg19 sequence, but is not limited thereto.

In the present invention, the BWA algorithm may include BWA-ALN, BWA-SW, Bowtie2 or the like, but is not limited thereto.

In the present invention, step c) of detecting the quality of the aligned reads means detecting how much the actual sequencing read matches the reference genome database using a mapping quality score.

In the present invention, step c) is carried out through a process including:

(c-i) specifying a region of each aligned nucleic acid sequence; and

(c-ii) selecting a sequence satisfying a cut-off value of a mapping quality score and a cut-off value of a GC ratio within the region.

In the present invention, in step (c-i) of specifying the region of the nucleic acid sequence, the region of the nucleic acid sequence may have a length of 20 kb to 1 Mb, but is not limited thereto.

In the present invention, in step (c-ii), the cut-off value may vary depending on the desired degree of the mapping quality score, but is specifically 15 to 70, more specifically 30 to 65, and most specifically 60. In step (c-ii), the GC ratio may vary depending on the desired degree of the GC ratio, but is specifically 20 to 70%, and more specifically 30 to 60%.

In the present invention, step c) may be performed excluding data of the centromere or the telomere of the chromosome.

As used herein, the “centromere” may have a length of about 1 Mb from the starting point of each chromosome long arm (q arm), but is not limited thereto.

As used herein, the “telomere” may have a length of about 1 Mb from the starting point of each chromosome short arm (p arm) or about 1 Mb from the ending point of each chromosome long arm (q arm), but is not limited thereto.

In the present invention, step d) is carried out through a process including:

(d-i) segmenting the reference genome into predetermined bins;

(d-ii) calculating a number of reads aligned in each bin and an amount of GC of the reads;

(d-iii) performing a regression analysis based on the number of reads and the amount of GC to calculate a regression coefficient; and

(d-iv) normalizing the number of reads using the regression coefficient.

In the present invention, the predetermined bin in step (d-i) may be 100 kb to 2,000 kb in length.

In the present invention, in step (d-i) of segmenting the reference genome into predetermined bins, the predetermined bin is 100 kb to 2 Mb, specifically 500 kb to 1500 kb, more specifically 600 kb to 1600 kb, more specifically 800 kb to 1200 kb, most specifically 900 kb to 1100 kb, but is not limited thereto.

In the present invention, the regression analysis in step (d-iii) may be any regression analysis method capable of calculating a regression coefficient, and is specifically a LOESS analysis, but is not limited thereto.

In the present invention, step e) of calculating the Z score may include standardizing the sequencing read value in each specific bin, and the calculation may be specifically carried out using Equation 1 below.

$$Z score = \frac{\begin{matrix}Read value of sequence information \\ sample of biological specimen - \\ Mean sequence information \\ read value of reference group\end{matrix} }{\begin{matrix}Standard deviation of mean sequence \\ information read value of reference group\end{matrix} } \qquad [ Formula 1 ]$$

In the present invention, step (f) includes:

(f-i) segmenting a chromosome region using circular binary segmentation (CBS) based on a Z score in each bin;

(f-ii) obtaining a chromosome length (size) of an area where a mean absolute value of a Z score of the segmented region is greater than or equal to a cut-off value; and

(f-iii) calculating an I-score in accordance with the following Formula 2:

:I=Σ_{j from all segmented above absolute mean Z score value 2}^{□}|MeanZⱼ|*Sizeⱼ. [Formula 2]

In the present invention, the cut-off value of the mean absolute value of the Z score is 1 to 2, and more specifically, 2.

In the present invention, the CBS algorithm refers to a method of detecting the point at which a change in the Z score, calculated in the step described above, occurs.

That is, the following formula is satisfied under the condition of 1<=i<j<=N on the assumption that i is the point at which the change of the Z score of the chromosome begins, j is a point at which the change of the Z score of the chromosome ends, N is the total length of the region, r is the bin value of each nucleic acid sequence (specific bin), and s is a standard deviation of bins.

$$\begin{matrix}{S}_{i} = {r}_{1} + {r}_{2} + … + {r}_{i} & [ Formula 6 ] \\ {S}_{j} = {r}_{1} + {r}_{2} + … + {r}_{j} & [ Formula 7 ] \\ {S}_{{i}_{j}} = {S}_{j} - {S}_{i} = {\sum }_{n = i + 1}^{j} {r}_{n} & [ Formula 8 ] \\ {T}_{ij} = ( \frac{{S}_{ij}}{j - 1} - \frac{{S}_{j - i} - {S}_{ij}}{N - j - i} ) / ( S \sqrt{\frac{1}{j - i} + \frac{1}{N - j - i}} ) & [ Formula 9 ] \\ ( {i}_{c} , {j}_{c} ) = arg max \vert {T}_{ij} \vert & [ Formula 10 ]\end{matrix}$$

Here, (i_{c}, j_{c}) represents a location at which the Z score change actually occurred, max represents a maximum value, and arg means a declination.

In the present invention, the cut-off value of the I score may be 1637.

In the present invention, the method may further include measuring a concentration of the isolated cell-free DNA and determining a case where the concentration of the cell-free DNA is higher than a cut-off value to be a bad prognosis.

In the present invention, the cut-off value of the isolated cell-free DNA concentration may be 0.71 ng/μl.

In the present invention, the method further may include classifying a case where the I score is 1638 to 3012 as a moderate risk group, classifying a case where the I score is 3013 to 13672 as a high risk group, and classifying a case where the I score is 13673 to 28520 as an ultra-high risk group.

In another aspect, the present invention is directed to a device for determining a prognosis of liver cancer based on cell-free DNA (cfDNA), the device including: a decoder for decoding reads (sequence information) of cell-free DNA isolated from a biological sample; an aligner for aligning the decoded reads to a reference genome database of a reference group; a quality controller for selecting only reads having a quality equal to or higher than a cut-off value from the aligned reads; and a determiner for calculating a Z score through comparison of selected reads with a reference group sample, calculating an I score based on the Z score and determining that the prognosis of liver cancer is bad when the resulting I score is higher than a cut-off value.

In the present invention, the cut-off value of the I score may be 1637.

In the present invention, the device may further include a concentration-based prognosis determiner for measuring a concentration of the isolated cell-free DNA and determining that the prognosis is bad when the concentration of the cell-free DNA is higher than a cut-off value.

In the present invention, the cut-off value of the concentration of the isolated cell-free DNA may be 0.71 ng/μl.

In another aspect, the present invention is directed to a computer-readable medium including an instruction configured to be executed by a processor for determining a prognosis of liver cancer, the computer-readable medium including: a) obtaining reads (sequence information) of cell-free DNA isolated from a biological sample; b) aligning the reads to a reference genome database of a reference group; c) detecting a quality of the aligned reads and selecting only reads having a quality equal to or higher than a cut-off value; d) segmenting the reference genome into predetermined bins, and detecting and normalizing amounts of the selected reads in the respective bins; e) calculating a mean and a standard deviation of normalized reads matched to each bin of the reference group and then calculating a Z score from normalized values in step d); f) segmenting chromosome using the Z score and calculating an I score; and g) determining that a prognosis of liver cancer is bad when the resulting I score is higher than a cut-off value.

In the present invention, the cut-off value of the I score may be 1637.

In the present invention, the computer-readable medium may further include measuring the concentration of the isolated cell-free DNA and determining that the prognosis is bad when the concentration of the cell-free DNA is higher than a cut-off value.

In the present invention, the cut-off value of the concentration of the isolated cell-free DNA may be 0.71 ng/μl.

In another aspect, the present invention is directed to a method of providing information for determining the prognosis of liver cancer including the method.

In the present invention, the liver cancer may be any type of cancer that occurs in the liver, and is not particularly limited and more specifically includes hepatocellular carcinoma (hepatocellular carcinoma with or without fibrous lamella deformation), cholangiocarcinoma (intrahepatic gallbladder duct carcinoma), and combined hepatocellular-cholangiocarcinoma, but is not limited thereto.

As used herein, the term “prognosis” means the prediction of the progression of cancer, recurrence of cancer and/or the possibility of metastasis of cancer. The prediction method of the present invention can be used to make a decision on clinical treatment by selecting the most appropriate treatment method for any particular patient. The prediction method of the present invention is a valuable tool for diagnosis regarding the determination as to whether or not the progression of cancer, recurrence of cancer and/or the possibility of metastasis of cancer of a patient are likely to occur, and/or for assisting in diagnosis.

### EXAMPLE

Hereinafter, the present invention will be described in more detail with reference to examples. However, it will be obvious to those skilled in the art that these examples are provided only for illustration of the present invention, and should not be construed as limiting the scope of the present invention.

### Example 1. Calculation of I-Score in Liver Cancer Patients and Normal Subjects

Cell-free DNA was extracted from plasma samples of 151 liver cancer patients and from plasma samples of normal subjects, and a library of full-length chromosomes was produced. The extraction of cell-free DNA was performed in the following process: 1) Separation of supernatant (plasma) by sequential centrifugation at 1,600 g for 10 minutes and 3,000 g for 10 minutes within 4 hours after collection of blood in an EDTA Tube; 2) extraction of cell-free DNA from 1.5 ml of the separated plasma using a QIAamp circulating nucleic acid kit; and 3) reaction of the final extracted cell-free DNA with a Qubit 2.0 Fluorometer and measurement of the concentration (ng/μl); and the library was prepared using a Truseq nano kit from Illumina, and a total of 5 ng of cell-free DNA was used for the reaction. Table 1 shows the information of 151 liver cancer patients who participated in this study.

TABLE 1
Clinical information of 151 liver cancer patients

| Characteristics | N = 151 |
|---|---|
| Age, years | 57 (52-63) |
| Sex | |
| Male | 137 (90.7%) |
| Female | 14 (9.3%) |
| ECOG performance status | |
| 0 | 52 (34.4%) |
| 1 | 97 (64.2%) |
| 2 | 2 (1.3%) |
| Etiology | |
| Hepatitis B | 134 (88.7%) |
| Hepatitis C | 4 (2.6%) |
| Alcohol | 7 (4.6%) |
| Others | 6 (4.0%) |
| Child-Pugh class | |
| A | 140 (92.7%) |
| B | 11 (7.3%) |
| BCLC stage | |
| B | 5 (3.3%) |
| C | 146 (96.7%) |
| Macrovascular invasion | |
| Yes | 63 (41.7%) |
| No | 88 (58.3%) |
| No. of extrahepatic spread organ sites | |
| 0 | 16 (10.6%) |
| 1 | 78 (51.7%) |
| 2 | 41 (27.2%) |
| ≥3 | 16 (10.6%) |
| Sites of extrahepatic spread | |
| Lymph node | 64 (42.4%) |
| Lung | 77 (51.0%) |
| Bone | 32 (21.2%) |
| Peritoneum | 23 (15.2%) |
| Adrenal gland | 13 (8.6%) |
| Others? | |
| AFP (ng/mL) | |
| <20 | 41 (27.1%) |
| 20-200 | 32 (21.2%) |
| >200 | 77 (51.0%) |
| Not available | 1 (0.7%) |
| Platelet count (×10³/mm³) | 122.0 (85.0-165.0) |
| Prothrombin time (INR) | 1.08 (1.02-1.16) |
| Albumin (g/dL) | 3.7 (3.4-4.0) |
| Total bilirubin (mg/dL) | 0.7 (0.5-1.0) |
| AST (IU/L) | 39 (28-58) |
| ALT (IU/L) | 26 (18-39) |
| Previous therapy | |
| No | 10 (6.6%) |
| Yes | 141 (93.4%) |
| Surgical resection | 69 (45.7%) |
| RFA | 37 (24.5%) |
| TACE | 118 (78.1%) |
| Radiotherapy | 79 (52.3%) |
| Liver transplantation | 12 (7.9%) |

Data are the median (Interquartile range) or number (%) unless otherwise indicated.
ECOG, Eastern Cooperative Oncology Group;
BCLC, Barcelona Clinic Liver Cancer;
AFP, alpha fetoprotein;
INR, international normalized ratio;
AST, aspartate aminotransferase;
ALT, alanine aminotransferase;
RFA, radiofrequency ablation;
TACE, transcatheter arterial chemoembolization.

The completed library was subjected to sequencing with NextSeq equipment, and sequence information data corresponding to a mean of 10 million reads (1 million reads-100 million reads) per sample was produced.

The Bcl file (including nucleotide sequence information) was converted to fastq format using the next-generation nucleotide sequencing (NGS) equipment, and the library sequence of the fastq file was aligned based on the reference genome Hg19 sequence using the BWA-mem algorithm. It was found that the mapping quality score satisfied 60.

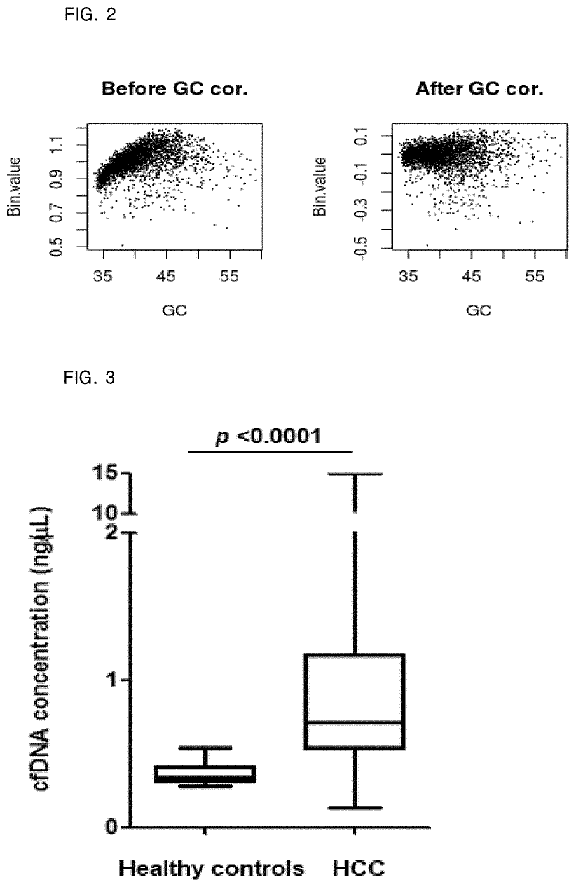

It was confirmed that the distribution of the number of sequencing reads in each chromosome locus bin was biased according to the amount of GC (FIG. 2), and the number of library sequences aligned according to the GC ratio in each chromosome was calibrated using regression analysis.

Then, the Z score was calculated using the following Formula 1:

$$Z score = \frac{\begin{matrix}Read value of sequence information \\ sample of biological specimen - \\ Mean sequence information \\ read value of reference group\end{matrix} }{\begin{matrix}Standard deviation of mean sequence \\ information read value of reference group\end{matrix} } \qquad [ Formula 1 ]$$

In order to calculate the I-score, first, chromosome was segmented using the CBS algorithm using the calculated Z score in each bin as data.

The mean Z score of the segmented area having a mean abstract Z score of 2 or more was multiplied by the chromosome length, and an I-score of each sample was obtained as the sum of the multiplied values. A sample in which the I-score was higher than 1637 was determined to be a sample in which the amount of cell-free DNA in the blood was high and the prognosis for Sorafenib treatment was bad. The I-score was calculated in accordance with the following Formula 2 below, and the I-scores (%) are shown in Table 2.

:I=Σ_{j from all segmented above absolute mean Z score value 2}^{□}|MeanZⱼ|*Sizeⱼ [Formula 2]

TABLE 2
Distribution (%) of I-scores of 151 liver cancer patients

| Liver cancer cohort (%) | I-score |
|---|---|
| 0~12.50% | 256~611 |
| 12.51%~25.00% | 612~762 |
| 25.01%~37.50% | 763~1003 |
| 37.51%~50.00% | 1004~1637 |
| 50.01%~62.50% | 1638~3012 |
| 62.51%~75.00% | 3013~7448 |
| 75.01%~87.50% | 7449~13672 |
| 87.51%~100.00% | 13673~28520 |

### Example 2. Confirmation of Effect of Blood-Cell-Free DNA Concentration (Ng/μl) on Progression of Liver Cancer and Survival

The distribution of cell-free DNA concentrations extracted from plasma of a total of 151 liver cancer patients ranged from 0.13 ng/μl to 15.00 ng/μl, and the median value thereof was 0.71 ng/μl. The distribution of cell-free DNA concentrations of 14 normal subjects ranged from 0.28 ng/μl to 0.54 ng/μl, and the median value thereof was 0.34 ng/μl. The test for the difference between the two groups was performed using the Mann-Whitney Test, and the result showed that there is a significant difference (p<0.0001) (FIG. 3).

The cell-free DNA concentration in blood also affected the prognosis (overall survival and time to progression) of 151 liver cancer patients. The risk of overall survival and time to progression was evaluated based on 0.71 ng/μl, which is the median blood-cell-free DNA concentration of the 151 patients. All 151 liver cancer patients took 400 mg of sorafenib twice a day, and the response to chemotherapy was evaluated every 6-8 weeks in accordance with RECIST guidelines Version 1.1.

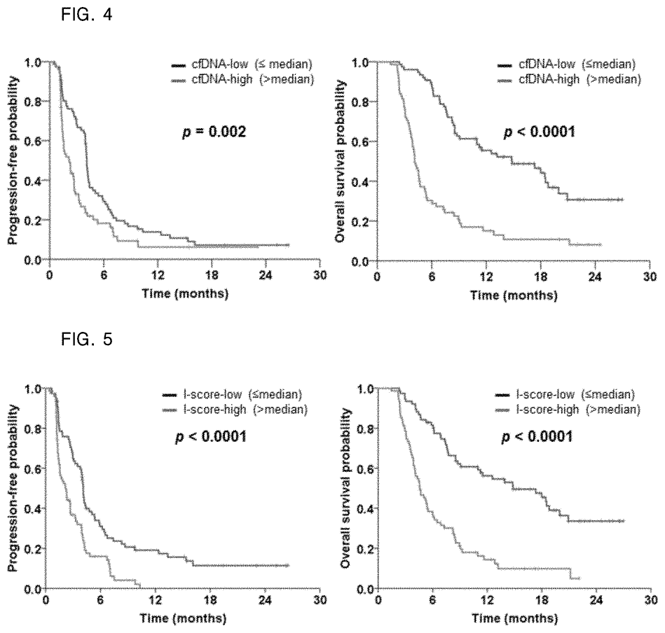

The result of the analysis showed that, when the cell-free DNA concentration was higher than 0.71 ng/μl, the hazard ratio (HR) regarding the time to progression was 1.71 (95% CI, 1.20-2.44; log-rank p=0.002), and the hazard ratio (HR) regarding the overall survival was 3.50 (95% CI, 2.36-5.20; log-rank p<0.0001). Based thereon, it was found that an increase in the blood concentration of cell-free DNA causes an increase in the risk of cancer progression and death (FIG. 4).

### Example 3. Confirmation of Effect of I-Score on Progression of Liver Cancer and Survival

The I-score of a total of 151 liver cancer patients ranged from 256 to 28,520, and the median value thereof was 1637. All 14 normal subjects had an I-score of 0 because no somatic CNA was found therein. The risk of overall survival and time to progression was evaluated based on the median I-score of 1637. All 151 liver cancer patients took 400 mg of sorafenib twice a day, and the response to chemotherapy was evaluated every 6-8 weeks in accordance with RECIST guidelines Version 1.1.

The result of analysis showed that, when the I-score was higher than 1637, the hazard ratio (HR) regarding the time to progression of the disease was 2.09 (95% CI, 1.46-3.00; log-rank p<0.0001), and the hazard ratio (HR) regarding survival was 3.35 (95% CI, 2.24-5.01; log-rank p<0.0001) (FIG. 5).

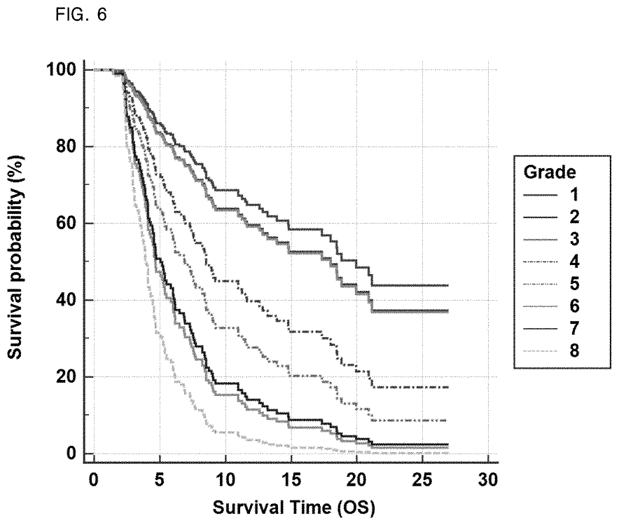

When the I-score is segmented on the basis of 8 grades, the hazard ratio regarding survival gradually increased in the order of 2.97 (95% CI, 1.28-6.90; p=0.01) for grade 5 (1638˜3012), 4.99 (95% CI, 2.19-11.41; p=0.0001) for grade 6 (3013˜7448), 4.52 (95% CI, 2.01-10.18; p=0.0003) grade 7 (7449˜13672), and 7.72 (95% CI, 3.31-18.02; p<0.0001) for grade 8 (13673˜28520) (FIG. 6).

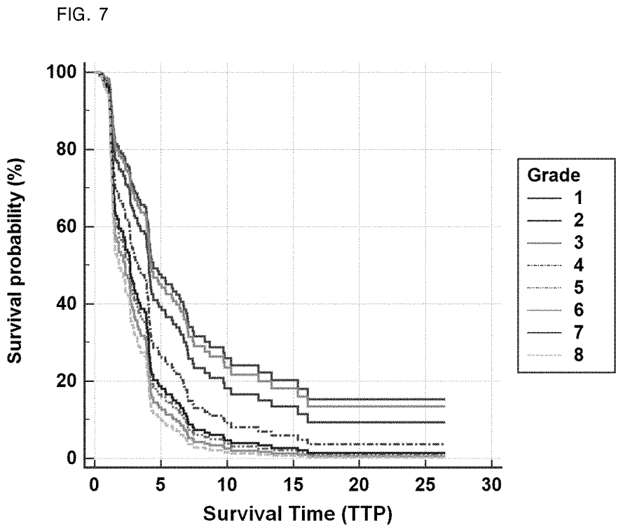

The hazard ratio (HR), which pertains to the time to progression, showed behavior similar thereto, and gradually increased in the order of 2.43 (95% CI, 1.21-4.86; p=0.01) for grade 5, 2.73 (95% CI, 1.36-5.48; p=0.0047) for grade 6, 2.26 (95% CI, 1.09-4.70; p=0.0294) for grade 7, and 3.08 (95% CI, 1.50-6.35; p=0.0022) for grade 8, which indicates that the risk of cancer progression increases as the I-score increases (FIG. 7).

This indicates that an increase in I-score causes an increase in the risk of cancer progression and death.

### Example 4. Confirmation of Correlation Between Cell-Free DNA Concentration and I-Score

As described above, the result of analysis showed that both blood-cell-free DNA concentration and I-score affect the progression of liver cancer and survival. Spearman correlation analysis was performed to determine the correlation between the two variables.

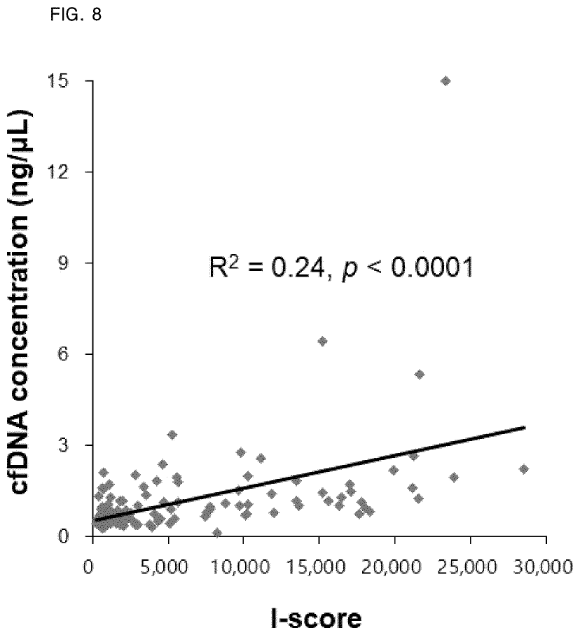

The result of analysis showed R²=0.24 and p<0.0001, which indicates that there is a direct correlation therebetween (FIG. 8).

Although specific configurations of the present invention have been described in detail, those skilled in the art will appreciate that this description is provided to set forth preferred embodiments for illustrative purposes and should not be construed as limiting the scope of the present invention. Therefore, the substantial scope of the present invention is defined by the accompanying claims and equivalents thereto.

### INDUSTRIAL APPLICABILITY

The method for determining the prognosis of liver cancer according to the present invention uses next-generation sequencing (NGS) and thereby is capable of improving the accuracy of prognostic prediction of liver cancer patients, as well as the accuracy of prognostic prediction based on cell-free DNA in a very low concentration, which has conventionally been difficult to detect, and of increasing commercial applicability. Therefore, the method of the present invention is useful for determining the prognosis of liver cancer patients.

1. A method of determining a prognosis of liver cancer based on cell-free DNA (cfDNA), the method including:
a) obtaining reads (sequence information) of cell-free DNA isolated from a biological sample;
b) aligning the reads to a reference genome database of a reference group;
c) detecting a quality of the aligned reads and selecting only reads having a quality equal to or higher than a cut-off value;
d) segmenting the reference genome into predetermined bins, and detecting and normalizing amounts of the selected reads in the respective bins;
e) calculating a mean and a standard deviation of normalized reads matched to each bin of the reference group and then calculating a Z score from normalized values in step d);
f) segmenting chromosome using the Z score and calculating an I score; and
g) determining that a prognosis of liver cancer is bad when the resulting I score is higher than a cut-off value.

2. The method according to claim 1, wherein step a) is carried out by a process comprising:
(a-i) removing proteins, fats and other residues from the isolated cell-free DNA using a salting-out method, a column chromatography method, or a bead method to obtain purified nucleic acids;
(a-ii) producing a single-end-sequencing or paired-end-sequencing library from the purified nucleic acids;
(a-iii) applying the produced library to a next-generation sequencer; and
(a-iv) obtaining reads of the nucleic acids from the next-generation sequencer.

3. The method according to claim 2, further comprising:
between the steps (a-i) and (a-ii), randomly fragmenting the nucleic acids purified in the step (a-i) by an enzymatic digestion, pulverization or HydroShear method to produce the single-end sequencing or paired-end sequencing library.

4. The method according to claim 1, wherein step a) of obtaining the reads comprises obtaining the isolated cell-free DNA through full-length genome sequencing with a depth of 1 million to 100 million reads.

5. The method according to claim 1, wherein step c) is carried out through a process comprising:
(c-i) specifying a region of each aligned nucleic acid sequence; and
(c-ii) selecting a sequence satisfying a cut-off value of a mapping quality score and a cut-off value of a GC ratio within the region.

6. The method according to claim 5, wherein the cut-off value of the mapping quality score is 15 to 70 and the cut-off value of the GC ratio is 30 to 60%.

7. The method according to claim 5, wherein step c) is performed excluding data of a centromere or a telomere of the chromosome.

8. The method according to claim 1, wherein step d) is carried out through a process comprising:
(d-i) segmenting the reference genome into predetermined bins;
(d-ii) calculating a number of reads aligned in each bin and an amount of GC of the reads;
(d-iii) performing a regression analysis based on the number of reads and the amount of GC to calculate a regression coefficient; and
(d-iv) normalizing the number of reads using the regression coefficient.

9. The method according to claim 8, wherein the predetermined bin in step (d-i) is 100 kb to 2,000 kb in length.

10. The method according to claim 1, wherein step e) of the calculation is carried out using Formula 1 below:
$$Z score = \frac{\begin{matrix}Read value of sequence information \\ sample of biological specimen - \\ Mean sequence information \\ read value of reference group\end{matrix} }{\begin{matrix}Standard deviation of mean sequence \\ information read value of reference group\end{matrix} } . \qquad [ Formula 1 ]$$

11. The method according to claim 1, wherein step (f) is carried out by a process comprising:
(f-i) segmenting a chromosome region using circular binary segmentation (CBS) based on a Z score in each bin;
(f-ii) obtaining a chromosome length (size) of an area where a mean absolute value of a Z score of the segmented region is greater than or equal to a cut-off value; and
(f-iii) calculating an I-score in accordance with the following Formula 2:
:I=Σ_{j from all segmented above absolute mean Z score value 2}^{□}|MeanZⱼ|*Sizeⱼ. [Formula 2]

12. The method according to claim 11, wherein the cut-off value of the mean absolute value of the Z score is 1 to 2.

13. The method according to claim 1, wherein the cut-off value of the I score is 1637.

14. The method according to claim 1, further comprising:
measuring a concentration of the isolated cell-free DNA and determining a case where the concentration of the cell-free DNA is higher than a cut-off value to be a bad prognosis.

15. The method according to claim 14, wherein the cut-off value of the isolated cell-free DNA concentration is 0.71 ng/μl.

16. The method according to claim 1, further comprising:
classifying a case where the I score is 1638 to 3012 as a moderate risk group, classifying a case where the I score is 3013 to 13672 as a high risk group, and classifying a case where the I score is 13673 to 28520 as an ultra-high risk group.

17. A method of providing information for determining a prognosis of liver cancer using the method according to claim 1.

18. A device for determining a prognosis of liver cancer based on cell-free DNA (cfDNA), the device comprising:
a decoder for decoding reads (sequence information) of cell-free DNA isolated from a biological sample;
an aligner for aligning the decoded reads to a reference genome database of a reference group;
a quality controller for selecting only reads having a quality equal to or higher than a cut-off value from the aligned reads; and
a determiner for calculating a Z score through comparison of selected reads with a reference group sample, calculating an I score based on the Z score and determining that the prognosis of liver cancer is bad when the I score is higher than a cut-off value.

19. The device according to claim 18, further comprising:
a concentration-based prognosis determiner for measuring a concentration of the isolated cell-free DNA and determining that the prognosis is bad when the concentration of the cell-free DNA is higher than a cut-off value.

20. A computer-readable medium comprising an instruction configured to be executed by a processor for determining a prognosis of liver cancer, the computer-readable medium comprising:
a) obtaining reads (sequence information) of cell-free DNA isolated from a biological sample;
b) aligning the reads to a reference genome database of a reference group;
c) detecting a quality of the aligned reads and selecting only reads having a quality equal to or higher than a cut-off value;
d) segmenting the reference genome into predetermined bins, and detecting and normalizing amounts of the selected reads in the respective bins;
e) calculating a mean and a standard deviation of normalized reads matched to each bin of the reference group and then calculating a Z score from normalized values in step d);
f) segmenting chromosome using the Z score and calculating an I score; and
g) determining that a prognosis of liver cancer is bad when the resulting I score is higher than a cut-off value.

21. The computer-readable medium according to claim 20, further comprising:
measuring a concentration of the isolated cell-free DNA and determining that the prognosis is bad when the concentration of the cell-free DNA is higher than a cut-off value.
